(19) United States
(12) Patent Application Publication
(10) Pub. No.: US 2022/0117768 A1
(43) Pub. Date: Apr. 21, 2022

(54) ANKLE BRACE WITH REAR HEEL CALCANEAL STABILIZING DEPRESSION

(71) Applicant: Royal Manufacturing LLC, Westfield, IN (US)

(72) Inventors: Richard JONES, Westfield, IN (US); Steven M. BUMGARDNER, Fort Wayne, IN (US); David COLLINS, Scarborough, ME (US); Lenny HOLDEN, Irvine, CA (US)

(73) Assignee: Royal Manufacturing LLC, Westfield, IN (US)

(21) Appl. No.: 17/503,461

(22) Filed: Oct. 18, 2021

Related U.S. Application Data
(60) Provisional application No. 63/093,580, filed on Oct. 19, 2020.

(51) Int. Cl. A61F 5/01

(52) U.S. Cl. CPC A61F 5/0111; A61F 2005/0181

(57) ABSTRACT

Ankle brace having a rubber stabilizer that fits under the heel of the wearer with uprights on either side. The rear bottom of the stabilizer has a semi-circular depressed area with a gradual taper in thickness that is thinnest along the rear-most edge and thickest where the semi-circular area connects to the interior of the stabilizer element. When weight is placed on the heel, the depression operates to leverage the front of the foot upward, which takes up slack in the ligaments of a person.

### CROSS REFERENCE TO RELATED APPLICATIONS

This application claims benefit of Provisional Application Ser. No. 40/883,185 filed Oct. 19, 2020, the disclosure of which is incorporated herein by reference in its entirety.

### FIELD OF THE INVENTION

The present invention relates to ankle braces.

### BACKGROUND

Most ankle sprains are probably self-treated and are never reported to a health care provider; therefore, many ankle sprains are not documented. Sprained ankles have been estimated to constitute up to 30% of injuries seen in sports medicine clinics and are the most frequently seen musculoskeletal injury seen by primary care providers. More than 23,000 people per day in the United States, including athletes and non-athletes, require medical care for ankle sprains. Stated another way, incident cases have been an estimated at 1 case per 10,000 persons per day. A U.S. Army study found that ankle sprains are the most common foot and ankle injury in active duty Army personal with a rate of 103 sprains per 1,000 persons per year.

Female athletes are 25% more likely to sustain ankle injuries than male athletes. Female basketball players are at a higher risk of a first-time inversion injury than those participating in other sports. Soccer and volleyball are other leading cases causes of ankle sprains in high school and college female athletes. Some studies attribute a higher incidence of ankle injuries in high school football, basketball, and soccer players. Other studies conclude that in college men, the risk of suffering an ankle sprain appears to be similar with basketball, soccer, and football.

A cohort study analyzed risk factors in ankle injuries from the Cadet Illness and Injury Tracking System (CIITS) DATABASE AT THE United States Military Academy (USMA) FROM 2005-2009. The results found higher risk of syndesmotic ankle sprains in males who performed at a higher level of athletic competition; male athletes were 3 times more likely to experience medial (eversion) sprains ten female athletes.

Ankle injuries are very common, with ankle sprain being the most common injury. Most individuals have complete recovery, however substantial number have chronic problems that usually lead to modification of physical activity.

Braces for feet and shows are disclosed, for example, in U.S. Pat. Nos. 660,885, 4,527,556, 4,998,537, 5,038,762, 5,113,877, 5,226,875, 5,449,005, 5,716,335, 5,741,222, 5,778,663, 6,022,331, 6,447,469, 6,945,947, 8,425,442, 8,622,946, US20050222531A1, US20070049855A1, US20090216167A1, US20160081838A1, U.S. D834205. U.S. Pat. Nos. 6,858,017, 6,749,578 and U.S. Re. 33,395.

Some of these braces can provide greater flexibility and comfort due to various features, such as being pivotable on both sides of the brace, which enabled the foot to flex forward and backward while limiting side-to-side motion of the foot relative to the leg in order to protect the ankle. Also, in some prior art designs, a semi-rigid stirrup encircles the bottom of the foot, interfering with a person's foot spreading out as he put his weight on the foot, thereby causing irritation and pain. If the stirrup were made wide enough to avoid that problem, it would provide less support to the person's ankle and might be too wide to fit into the person's shoe and that an improper brace function could cause harm.

However, shortcomings of some of these braces include, movement constriction, blood flow constriction, weakening of foot or ankle, sets the body up for other injuries and being unsuitable for rehabilitation. It would be desirable to provide an improved brace that does not constrict movement or blood flow and that strengthen the foot or ankle and promote rehabilitation. It would also be desirable to provide an improved brace that provides calcaneal stabilization/tilt when a person exerts weight in the heel.

### BRIEF DESCRIPTION OF THE DRAWINGS

FIG. 1 is a top view of a flattened version of the rubber foot stabilizer element showing the increased taper of the element in the area where it will abut the back of the heel of a user, an inset in the rear edge of the stabilizer such that it is narrowest at its midline, and the teardrop-shaped recesses in the upper arms that receive the plastic stiffener.

FIG. 2 is a bottom view of a flattened version of the rubber foot stabilizer element.

FIG. 3 is a cross-sectional view of the foot stabilizer element along line A1-A2 as shown in FIG. 1 showing that the stabilizer element has an increased taper in the rear portion, and a consistent thickness in its middle and front areas.

FIG. 4 is a cross-sectional of the foot stabilizer element along line B1-B2 as shown in FIG. 1 showing that the stabilizer has a taper around its perimeter, and a consistent thickness in its interior area.

FIG. 5 is a cross-sectional view of the foot stabilizer element along line C1-C2 as shown in FIG. 1 showing that the stabilizer has a taper around its perimeter, a recess, an embedded stiffener within the recess, and a cushioning material.

FIG. 6 is a cross sectional view of a foot that is wearing the ankle brace showing the increased taper of the stabilizer element from the center of the heel toward the back of the heel when weight is not being placed on the heel, such that there is a gap between the back of the heel and the tapered semi-circular back area of the stabilizer element.

FIG. 7 is a cross sectional view of a foot that is wearing the ankle brace showing the increased taper of the stabilizer element from the center of the heel toward the back of the heel when weight is being placed on the heel, such that there is not a gap between the back of the heel and the tapered semi-circular back area of the stabilizer element so that the stabilizer element leverages the front of the foot upward.

FIG. 8 is a bottom view of a foot wearing the brace showing the location of the tapered semi-circular back area of the stabilizer element with respect to the foot.

FIG. 9 is a perspective view of the ankle brace including both the stabilizer element and the woven sock to which it is cemented.

FIG. 10 is an exploded view of an embodiment of the invention showing how the components may be assembled.

### DESCRIPTION OF THE INVENTION

The invention comprises a rubber stabilizer that goes under the heel and midfoot of the wearer. The stabilizer has uprights on either side. Significantly, the rear bottom of the stabilizer has a semi-circular area with a gradual taper in thickness that is thinnest along the rear-most edge and thickest where the semi-circular area connects to the interior of the stabilizer element. The stabilizer is cemented to a woven sock that receives the foot of a person.

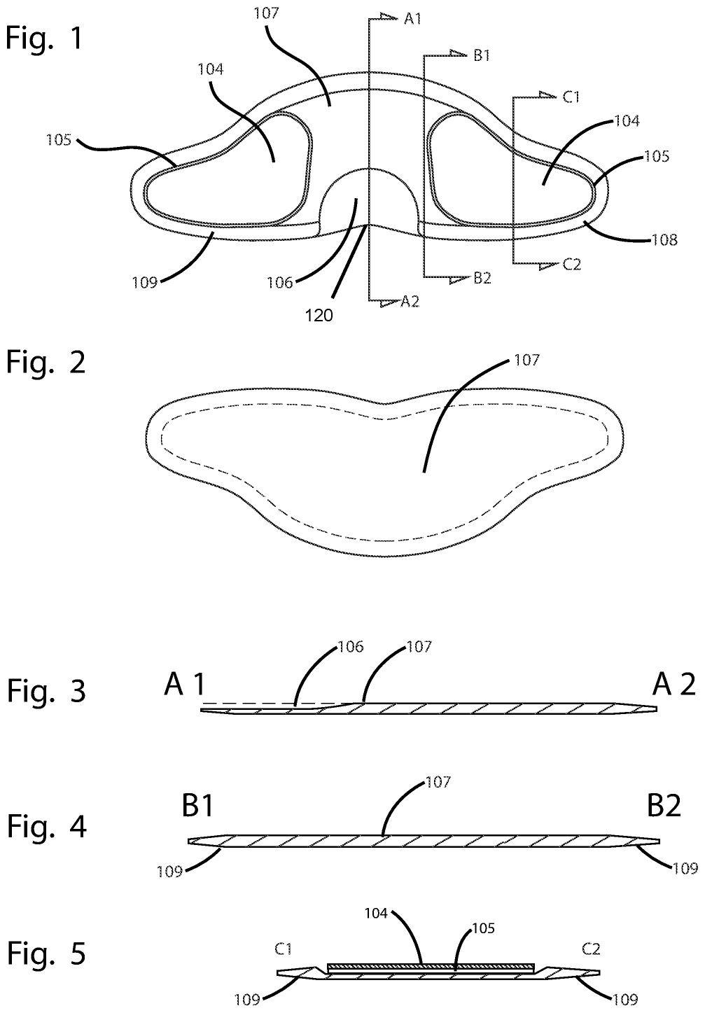

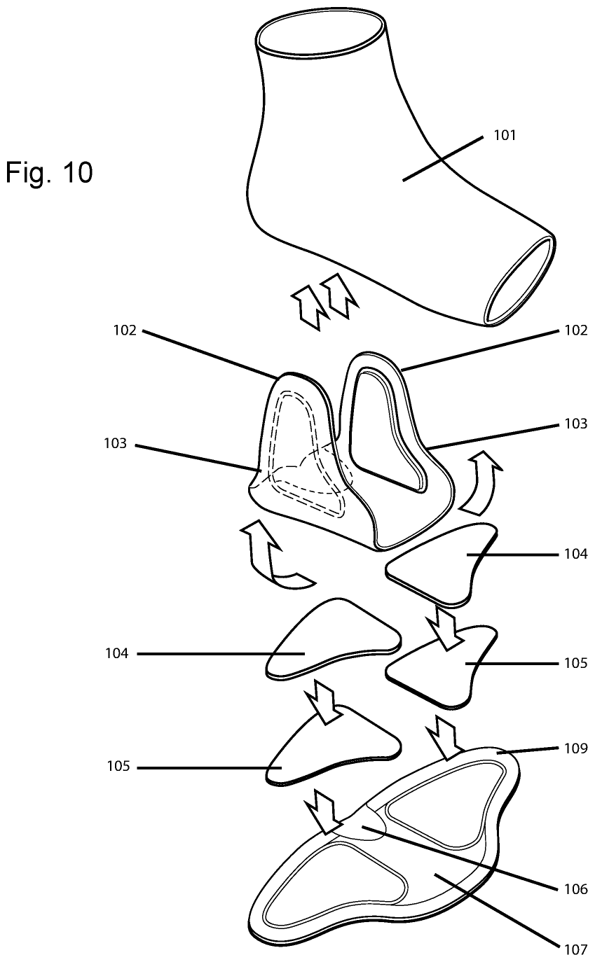

FIG. 1 is a top view of a flattened version of the rubber foot stabilizer element 107 showing the increased taper of the element or a depression in the area 106 where it will abut the back of the heel of a user, an inset 120 in the rear edge of the stabilizer such that it is narrowest at its midline, and the teardrop-shaped recesses in the upper arms that receive plastic stiffeners 105, which in turn are cemented to tear-drop shaped cushions 104 as further shown in FIG. 10. Stabilizer element may be from 3 mm-10 mm thick, except for its perimeter edge 109 which is about 10 mm wide and that may taper from the central portion to its outer edge where it is 1 mm or less thick. In one embodiment, when flattened as shown in FIGS. 1 and 2 the stabilizer element is about 381.5 mm wide and about 134 mm. from front to back.

Rubber foot stabilizer element 107 includes a bottom portion upwardly projecting arms, which are on the right and left sides as shown in FIGS. 1 and 2, but shown in their upward positions in FIG. 9. Each upwardly projecting arm is tapered to be wider at its bottom 103 and narrowing to a rounded top portion 102. Each upwardly extending arm also comprises a teardrop-shaped recess in its rubber thickness that is wider at its bottom than at its top as best shown by the dashed lines in FIG. 9. Each arm also has a teardrop-shaped plastic stiffener 105 having an outer edge within the recess. The stiffeners 105 may be formed form thermoformed plastic sheet and cemented to the rubber stabilizer element. Teardrop-shaped cushions 104 having outer edges that are slightly smaller than the stiffeners 105 are placed over the plastic stiffeners 105 such that the outer edge of the plastic stiffeners 105 are exposed. These cushions 104 may be comprised of ethyl vinyl acetate and be about 2.5 mm thick.

The rubber stabilizer element including the plastic stiffeners and cushions may be cemented using a non-acetate cement or silicone to sock 101 to form the complete brace as shown in FIG. 9. The sock 101 may be comprised of a cotton or cotton-poly blend.

FIG. 2 is a bottom view of a flattened version of the rubber foot stabilizer element 107. The perimeter is shown by the dashed line.

FIG. 3 is a cross-sectional view of the foot stabilizer element along line A1-A2 as shown in FIG. 1 showing that the stabilizer element has an increased taper (or decreased thickness) in the rear portion, and a consistent thickness in its middle and front areas.

The bottom portion of the stabilizer element is characterized by a tapered semi-circular shaped depressed area 106 in which the curved edge of the semi-circular shape has the same thickness as the central portion 107 of the stabilizer element and that tapers in thickness toward the back edge of the stabilizer element. The tapered semi-circular shaped depressed area 106 also include an inset 120 in the rear edge of the stabilizer such that it is narrowest at its midline.

FIG. 4 is a cross-sectional of the foot stabilizer element 107 along line B1-B2 as shown in FIG. 1 showing that the stabilizer has a taper around its perimeter 109, and a consistent thickness in its interior area across this cross-sectional line.

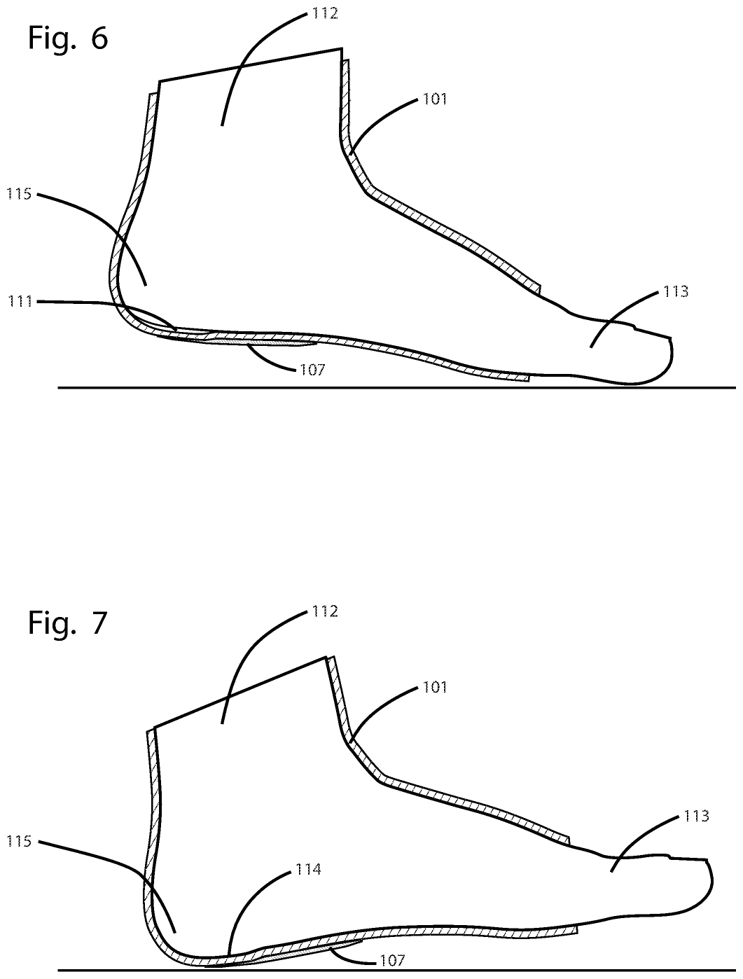

FIG. 5 is a cross-sectional view of the foot stabilizer element 107 along line C1-C2 as shown in FIG. 1 showing that the stabilizer has a taper around its perimeter 109, a recess, an embedded stiffener 105 within the recess, and a cushioning material 104.

FIG. 6 is a cross sectional view of a foot that is wearing the ankle brace showing the increased taper of the stabilizer element from the center of the heel toward the back of the heel when weight is not being placed on the heel, such that there is a gap 111 between the back of the heel 115 and the tapered semi-circular back area 106 of the stabilizer element 107.

FIG. 7 is a cross sectional view of a foot that is wearing the ankle brace showing the increased taper of the stabilizer element from the center of the heel toward the back of the heel when weight is being placed on the heel, such that there is no a gap 114 between the back of the heel 115 and the tapered semi-circular back area 106 of the stabilizer element 107 so that the stabilizer element leverages the front of the foot upward. This operates to take up slack in the calcanealfibular ligament, which attaches the lateral side of the calcaneous to the fibia, the anterior talofibular ligament which attaches the fibia and the talus, and the anterior tiofibular ligament which attaches the fibia and tibia.

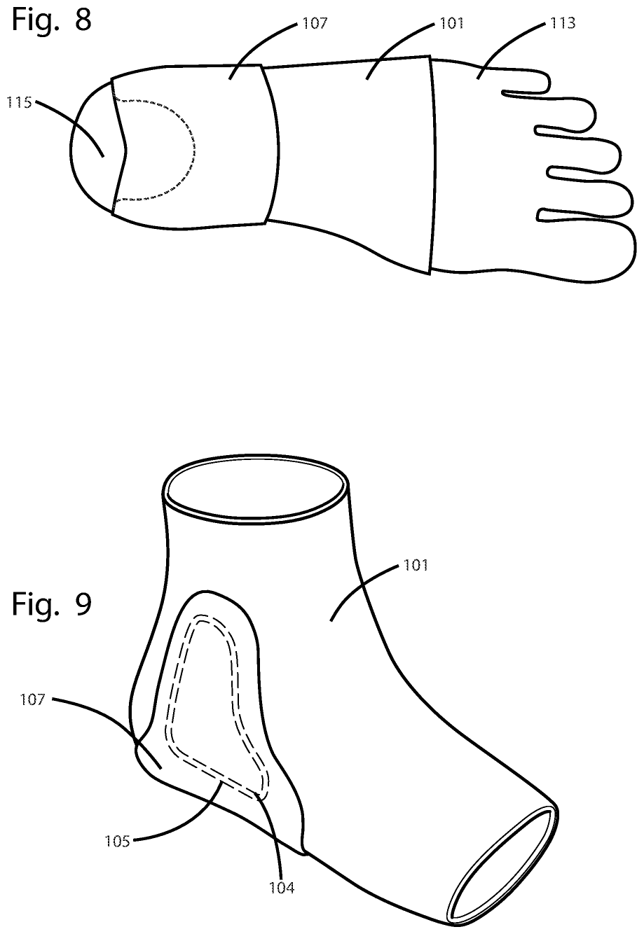

FIG. 8 is a bottom view of a foot wearing the brace showing the location of the tapered semi-circular back area of the stabilizer element (shown by dashed lines) with respect to the heel 115 of a foot 113.

FIG. 9 is a perspective view of the ankle brace including sock 101, stabilizer element 107, stiffener 105 and cushion 104. As shown by the dashed lines, the stiffener 105 and cushion 104 are on the side of stabilizer element toward the foot, as opposed to the exterior of the brace.

FIG. 10 is an exploded view of an embodiment of the invention showing how the components may be assembled. Stiffeners 105 are cemented into the recesses in stabilizer element 107, cushions 104 are cemented to stiffeners 104. Cement is the applied to these elements and the arms are bent upwardly to attach to sock 101.

Use of the ankle brace as shown and described promotes an improved leg orientation and during physical activity, reduced the risk of injury, and accelerates the healing of an injured foot, ankle or leg.

In an alternate embodiment, the stabilizer may also be formed with a side-to-side the pitch ranging from 1 to 10 mm, meaning the thickness of the midfoot stabilizer may be 0.05-3 mm on the medial side, and 0.15-13 mm on the lateral side. The exact pitch for a person may be selected taking into account the physiology of the person and if injured, the nature of the injury.

In an alternate embodiment, the brace may comprise a stirrup-shaped brace, optionally built into a shoe, boot or other footwear, that envelopes the mid and hindfoot to create support/control of over-exertion of the ankle in two ways. First, upward extension coupled with shoe provide a stabilizing effect on the joints comprising the ankle. The front and rear counters create support to assist with limiting the adverse effects of low, mid and high arch. Second, the bottom of the ASF extends rearward to the bony prominence at a pitch or before (such as 2 degrees) bevel that increases frontal calcaneal pitch, creating a lift in the arch of the foot without arch pressure and extends to behind metatarsal heads on a limited bias with the lateral extension forward assisting with locking the forefoot into a more rigid lever for propulsion. The Benefit of this pitch helps the ankle and foot to react quicker to help move the lateral or medial roll in the opposite direction thus helping to keep everything balanced and stable to help prevent an ankle injury from this enhancement of propulsion.

The stabilizer may be formed as either a stand-alone ankle brace, or be built into a shoe, boot or other footwear. The portion having the pitch may also have uprights that extend upward along the right and lefts side of the foot that are moldable, so that they conform to the contours of the wearer's ankle.

In an alternate embodiment a brace may be enclosed in a high top shoe, with a pliable stirrup brace extending from above lateral malleolus flaring posterior and anterior to mid calcaneus and anterior to mid lateral 5th metatarsal, wrapping under plantar surface of foot from calcaneal tuberosity posterior to anterior 5th metatarsal head on a bias posteriorly to mid, 1st metatarsal then again flaring posterior and anterior medially to mid calcaneus and mid, 1st metatarsal then upward over medial malleolus.

The present invention controls over eversion and over inversion of the ankle mortise, limiting stress on the Calcaneofibular ligament, Anterior talofibular ligament and Interosseous ligaments with the possibility of reducing high ankle sprains affecting above mentioned ligaments and strain to Peroneus tertius tendon and Peroneus brevis tendon in inversion type sprains/strains. In one embodiment, the posterior plantar surface of device is beveled at 2 degrees to increase anterior calcaneal pitch increasing uplift of foot arch without applying arch pressure. This calcaneal pitch will possibly tighten above-described ligaments.

Those of skill in the art will understand that various details of the invention may be changed without departing from the spirit and scope of the invention. Furthermore, the foregoing description is for illustration only, and not for the purpose of limitation, the invention being defined by the claims.

While the invention has been illustrated and described in detail in the foregoing drawings and description, the same is to be considered as illustrative and not restrictive in character, it being understood that only illustrative embodiments thereof have been show and described and that all changes and modifications that are within the scope of the following claims are desired to be protected.

All references cited in this specification are incorporated herein by reference to the extent that they supplement, explain, provide a background for or teach methodology or techniques employed herein.

We claim:

1. An ankle brace comprising:
a stabilizer element comprised of rubber, and that has
a bottom portion
a perimeter edge
a thickness
opposing upwardly extending arms that:
are tapered to be wider at the bottom where they join the bottom portion
have rounded top portions
and wherein each upwardly extending arm comprises
a teardrop-shaped recess in its rubber thickness that is wider at its bottom than at its top;
a teardrop-shaped plastic stiffener having an outer edge within the recess, and
a teardrop-shaped cushion having an outer edge placed over the plastic stiffener such that the outer edge of the plastic stiffener is exposed;
and wherein each upwardly extending arm has an upper area and a lower area, wherein the lower area is wider than the upper area;
and wherein the bottom portion is characterized by
a central area from 3 mm to 10 mm thick;
a tapered semi-circular shaped area in which the curved edge of the semi-circular shape has the same thickness as the central portion of the stabilizer element and that tapers in thickness toward the back edge of the stabilizer element; and
an inset in the rear edge of the stabilizer such that it is narrowest at its midline;
and wherein outermost edge of the stabilizer element has a thickness that is tapered such that the thickness of the outermost edge is less than 1.1 mm;
a woven sock having an open toe, and a bottom area to receive a heel, and wherein the stabilizer element is cemented to the sock such that the upwardly extending arms cover the ankle area of the sock and such that the innermost area of the semicircular shaped area is beneath the middle of the heel area.
